(19) United States
(12) Patent Application Publication
(10) Pub. No.: US 2010/0286549 A1
(43) Pub. Date: Nov. 11, 2010

(54) System and Method for Assessing Efficacy of Therapeutic Agents

(75) Inventors: Erwin Roy John, Mamaroneck, NY (US); Leslie S. Prichep, Mamoreneck, NY (US)

Correspondence Address: FAY KAPLUN & MARCIN, LLP, 150 BROADWAY, SUITE 702, NEW YORK, NY, 10038, US

(73) Assignee: New York University, New York, NY (US)

(21) Appl. No.: 12/744,549

(22) PCT Filed: Dec. 12, 2008
(86) PCT No.: PCT/US08/86575

§ 371 Date: Jul. 15, 2010

Related U.S. Application Data
(60) Provisional application No. 61/014,611, filed on Dec. 18, 2007.

(51) Int. Cl. A61B 5/0476; G06F 19/00

(52) U.S. Cl. 600/544; 702/19

(57) ABSTRACT

A method for assessing an effect of a therapeutic agent, comprises the steps of detecting brain electrical activity of a subject to generate a first set of brain wave data and extracting from the first set of brain wave data first data features sensitive to a neurological disorder in combination with the steps of comparing the first data features to control data to define a baseline profile of brain electropathophysiology and computing one of a first classifying score and a first discriminant score based on the baseline profile to estimate a probability that the baseline profile corresponds to a predetermined pathophysiological condition.

### PRIORITY CLAIM

This application claims priority to U.S. Provisional Application Ser. No. 61/014,611, entitled “System and Method for Assessing Efficacy of Therapeutic Agents” filed Dec. 18, 2007. The specification of the above-identified application is incorporated herewith by reference.

### FIELD OF THE INVENTION

The present invention related to a system and method for assessing the effectiveness of a therapeutic agent in a patient.

### BACKGROUND

Medications are typically prescribed for developmental, neurological or psychiatric disorders based upon a physician's medical opinion and experience with the disorder. The physician selects a treatment protocol (e.g., the medication(s) and dosage) based on a combination of objective and subjective symptoms exhibited by the patient. After the patient has taken the medication for a preidentified period of time (i.e., time sufficient for the medication to take effect), the physician evaluates the therapeutic effect of the treatment protocol and may adjust the dosage or select a different or additional medication.

### SUMMARY OF THE INVENTION

The present invention is directed to a method for assessing an effect of a therapeutic agent, comprising the steps of detecting brain electrical activity of a subject to generate a first set of brain wave data and extracting from the first set of brain wave data first data features sensitive to a neurological disorder in combination with the steps of comparing the first data features to control data to define a baseline profile of brain electropathophysiology and computing one of a first classifying score and a first discriminant score based on the baseline profile to estimate a probability that the baseline profile corresponds to a predetermined pathophysiological condition.

The present invention is further directed to a method for assessing the efficacy of a therapeutic agent comprising the steps of: collecting brain electrical activity data during an initial examination and subjecting the brain electrical activity data to spectral analysis; extracting from the brain electrical activity data a set of descriptors; comparing the descriptors to stored normative data to compute standard scores defining a “baseline profile” of brain electrical pathophysiology; using the baseline profile to compute one of a classifying score and a discriminant score to estimate a probability that the baseline profile corresponds to a specific pathophysiological condition associated with a disorder with which the patient has been diagnosed. The method according to the present invention may further include storing the baseline profile and the discriminant score with unique identifiers enabling future retrieval; selecting a preferred therapeutic agent by one of relying upon the medical personnel clinical judgment and a comparison of one of the baseline profile and the discriminating features to control data; administering a “test dose” of the preferred therapeutic agent; and after a predetermined interval has elapsed since administration of the test dose, collecting a second sufficient sample of brain electrical activity data, the predetermined interval being based on pharmacokinetic considerations; extracting a second set of descriptors and computing standard scores based thereon to generate one a post-treatment profile (e.g., including a discriminant score); and comparing the baseline profile to the post-treatment profile to evaluate the efficacy of the therapeutic agent.

### BRIEF DESCRIPTION OF THE DRAWINGS

FIG. 1 shows an exemplary embodiment of a system for assessing efficacy of a therapeutic agent according to the present invention; and

FIG. 2 shows an exemplary embodiment of a method for assessing efficacy of a therapeutic agent according to the present invention.

### DETAILED DESCRIPTION

The present invention may be further understood with reference to the following description and the appended drawings, wherein like elements are provided with the same reference numerals. Although the present invention describes a system and method for assessing the efficacy of a therapeutic agent by analyzing brain wave data collected from an electroencephalogram (EEG), those skilled in the art will understand that other types of data relating to brain activity may be manipulated in a manner similar to that described herein to achieve the same results. Thus, the description of EEG and the specific descriptions of EEG features is illustrative only of an exemplary embodiment of the invention and should not be construed to limit the scope of this invention. Similarly, the efficacy of other treatment protocols (e.g., surgical procedures, alternative therapies, etc.) may be analyzed using the brain activity data in a similar manner.

As understood by those skilled in the art, an electroencephalogram (EEG) detects neurophysiological activity by measuring an intensity and pattern of electrical signals generated by the brain. Undulations in the electrical signals are typically referred to as brain waves. The EEG is a record comprising these undulating electrical signals and other electrical activity (e.g., noise, event-related transients, etc.). The EEG is typically used to assist in the diagnosis, in children and adults, of developmental, neurological and physiological disorders. According to exemplary embodiments of the present invention, data corresponding to brain activity (e.g., EEG data) is utilized to determine the efficacy of a treatment protocol and, in particular, a therapeutic effect of a therapeutic agent such as a pharmaceutical and/or a combination of therapeutic agents prescribed in the treatment protocol. That is, brain waves produced in patients which exhibit symptoms (objective and subjective) of developmental, neurological and physiological disorders deviate from reference norms (e.g., self- and/or population norms) in predictable ways. By quantitatively analyzing EEG data after use of the therapeutic agent(s), it may be determined whether the pharmaceutical is returning the EEG data to reference norms. The quantitative analysis may also suggest a change in the treatment protocol (e.g., the therapeutic agent or combination of agents to be administered, dosage, timing of doses, etc.). Although the exemplary embodiments are described with reference to EEG data, those of skill in the art will understand that data corresponding to brain activity obtained using other signal collection/processing methods may be utilized to determine the efficacy of a treatment protocol.

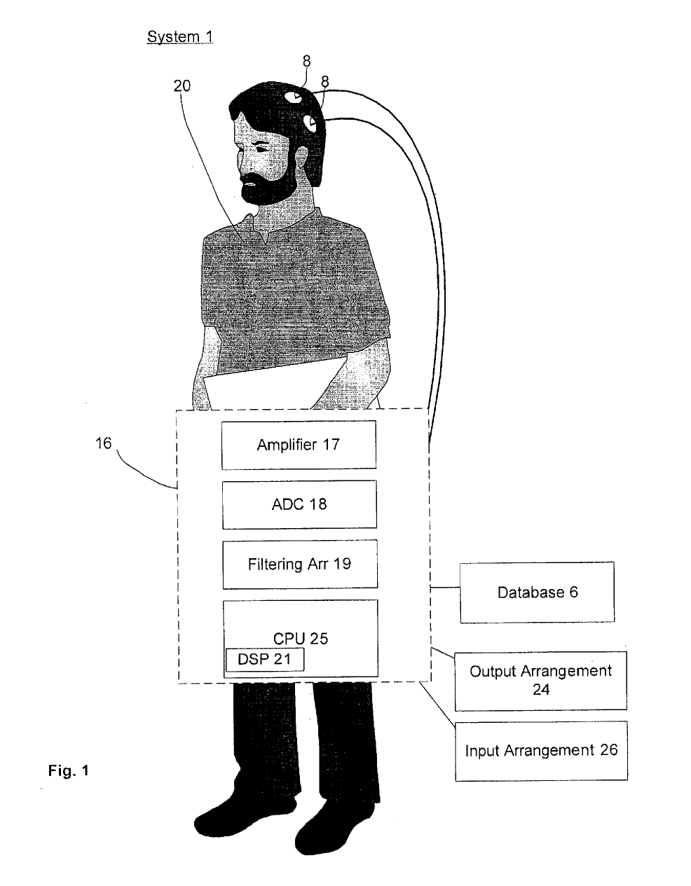

FIG. 1 shows an exemplary embodiment of a system 1 for assessing the efficacy of a therapeutic agent or agents according to the present invention. The system 1 includes a computing device 16 which harvests EEG data from a subject 20 with a developmental, neurological and/or physiological disorder after an administration of one or more therapeutic agents to determine whether the agents are returning the subject 20 toward EEG data indicative of a reference norm, e.g., a state without the developmental, neurological and physiological disorder or a more manageable form of the disorder. As described in more detail below, the EEG data is compared to control data which may be a self norm based on data obtained in the absence of symptoms or a population norm based on data from a population of individuals which do not exhibit symptoms of the disorder. In the exemplary embodiment, the device 16 is implemented as a portable, handheld device for use in a clinical or non-clinical setting. In an example of the latter case, the subject 20 may bring the device 16 home and allow the device 16 to collect the EEG data for a predetermined period of time which may be extended if desired as the subject is not required to stay in a hospital or treatment center. The EEG data may then be transmitted to the physician (e.g., via mail, email, etc.) for analysis.

The device 16 receives electrical signals corresponding to brain activity of the subject 20 from electrodes 8 attached to the subject's scalp, as would be understood by those skilled in the art. As will be described in more detail below, the electrical signals are converted into EEG data which is quantitatively analyzed in the device 16 to generate digital quantitative EEG (QEEG) data that is compared to control data (e.g., the self- and/or population-norms) stored, for example, in a database 6. As would be understood by those skilled in the art, the database 6 may be stored in a memory within the device 16 or may be in a remote storage accessed via, for example, a wireless or wired connection or may be partly stored within the device 6 and partly in a remote memory. The memory may, for example, be a removable item such as a memory card so that when the device 16 is used at home, only the memory card need be transferred to the physician. Alternatively, the memory may be permanently or temporarily stored in the device 16 using any of a wide range of memory devices including, for example, hard drives, solid state data storage chips, etc.

The reference norms in the database 6 correspond to (i) EEG data of individuals without (or a manageable form of) one or more target developmental, neurological and/or physiological disorders (e.g., the population norm) and (ii) EEG data of the subject 20 prior to administration of a prescribed therapeutic agent during, for example, a period when the subject is not showing symptoms of the target disorder(s) (e.g., the self norm). The database 6 may further include treatment data corresponding to EEG data of individuals with a developmental, neurological and/or physiological disorder, where one or more therapeutic agents have been administered for treatment and an outcome of the treatment is recorded. In this case, the device 16 may utilize the treatment data to suggest therapeutic agents based on the EEG data of the subject 20.

In the exemplary embodiment, the EEG data of the subject 20 is obtained after administration of one or more pharmaceuticals or other therapeutic agents and compared to reference norms in the database 6 to determine the efficacy of the agent(s). The EEG data may also be mapped onto the treatment data to determine a subsequent treatment protocol, e.g., change in type of medication, dosage adjustment, etc. That is, the EEG data of the subject 20 may be substantially similar to control data corresponding to a portion of the population from which the data was compiled (e.g., individuals with similar demographics, histories, etc.) so that treatment protocols associated with this portion of the population may be considered in adjusting/updating the treatment protocol of the subject 20.

As shown in FIG. 1, the device 16 is coupled to any number of EEG electrodes 8 which are applied to the scalp of a subject 20 or to an individual to be included in the control group in any known configuration. Those of skill in the art will understand that any conventional EEG biosensor electrodes may be used in conjunction with the present invention and these electrodes 8 may be either reusable (i.e., sterilizable) or disposable. For example, the electrodes 8 may be pre-gelled, self-adhesive disposable electrodes. Alternatively, the electrodes 8 may have multiple small barbs, a needle electrode or a conductive disc temporarily attached to the scalp. The electrodes 8 may also utilize conductive gel to provide rapid and secure attachment to the scalp while limiting noise. In other exemplary embodiments (e.g., a portable system), the electrodes 8 may be coupled to a cap placed on the head of the subject 20 and oriented to rest in desired positions relative to the scalp. Such a cap facilitates placement of the electrodes 8 in, for example, a home-use situation and reduces problems associated with the attachment of the electrodes 8 to the scalp. Thus, the device 16 may be configured to receive data from any number and/or type of biosensor electrodes and may be configured to separate data from groups of electrodes 8 allowing for simultaneous use with multiple patients. Such an arrangement may facilitate, for example, collection of the population norm and/or treatment data. Those skilled in the art will also understand that the device 16 may be used in conjunction with wired or wireless electrodes. In the case of wired electrodes, leads transfer the electrical signals to the device 16, whereas radio frequency signals may be used when the electrodes are wirelessly coupled to the device 16. In this embodiment, the electrodes are coupled to a radio frequency transmitters which transmit the signals to a receiver in the device 16 as would be understood by those skilled in the art.

The electrical signals from the electrodes 8 are transferred for processing to a high-gain, low-noise amplifier 17 in the device 16. The amplifier 17 may include an input isolation circuit to protect against current leakage, such as a photo-diode light-emitting diode isolation coupler and may be protected from electrical interference by a radio-frequency filter and/or a 60-cycle notch filter as would be understood by those skilled in the art. The signals output by the amplifier 17 are converted to digital signals by an analog-to-digital converter (ADC) 18 which samples at about 1 KHz; this may be downsampled to give a bandwidth of approximately 0 to 100 Hz.

These digital signals are transmitted to a digital signal processor (DSP) 21 which may be included in or electrically coupled to a central processing unit (CPU) 25. The DSP 21 utilizes a digital signal processing technique such as a Fast Fourier Transform (FFT), an Inverse Fast Fourier Transform (IFFT), a wavelet analysis, a principal component analysis, a logistic regression, a microstate analysis and wavelet denoising to compute a very narrow band (e.g., 0.5 Hz frequency intervals) power spectrum of the signals for each electrode over a bandwidth of interest (e.g., 0.5 to 100 Hz). Descriptors of the EEG, such as an absolute or a relative (e.g., a percentage) power of the EEG at every electrode, a gradient and a synchronization (e.g., coherence) of power between each electrode and every other electrode in the array, are extracted. The descriptors may be extracted for each frequency or selected combinations of frequencies (e.g., frequency bands). The set of such descriptive features obtained during an initial examination is compared to normative data stored in a database 6 or any other data storage structure, and each descriptor is resealed as a standard score (e.g., a Z-score).

The Z-scores are used to compute a “baseline profile”. The Z-scores may also be used to compute a discriminant score using a set of QEEG discriminant functions stored in the database 6, which estimate the probability that the observed profile was obtained from a patient afflicted with some disorder that is often associated with symptoms similar to those reported by the patient or a disorder for which the patient has been diagnosed. The baseline profile and/or the discriminant score may be stored in the database 6 for future reference, as described below.

A test dose of a therapeutic agent or other treatment may be determined by either comparing the baseline profile to a stored database of QEEG profiles or discriminant functions derived from patients with substantially similar symptoms or diagnoses who demonstrate a positive response to a particular therapeutic agent, or based on the clinical judgment of the responsible medical personnel. After a time interval considered adequate in view of the mode of administration and/or the known pharmacokinetics of the agent, a second sample of EEG is collected under the same conditions as the baseline sample. Using identical methods, a QEEG “post-treatment” profile and/or discriminant score is computed from the second EEG sample.

The post-treatment profile and/or the discriminant score are compared to the pre-treatment baseline profile or discriminant score, which are retrieved from storage after confirming their identity as belonging to the patient. As a result of the comparison, the CPU 25 outputs the differences between the QEEG features and/or discriminant scores before and after the test dose, in a form indicating whether the agent has achieved an improvement in the pathophysiological conditions or abnormal brain electrical activity reflected in the QEEG features, providing an estimation of the efficacy of the test dose in correcting electrophysiological correlates of the developmental, neurological and/or functional disorder of the patient 20. Analysis of the output will be described further below.

As would be understood by those skilled in the art, the device 16 may include or be coupled to one or more output arrangements 24. In the exemplary embodiment, the output arrangement 24 is a display screen which displays the QEEG profiles extracted from the baseline examination, the examination after the test dose, the differences between the pre and post treatment profiles of the patient 20 and their statistical significance. The normative values of the corresponding QEEG features may also be displayed. The screen may also display a graphical indication of the nature of the differences before and after treatment. This graphical indication might be a three-dimensional brain image color-coded to depict the severity of the QEEG abnormality, i.e., the values of the Z-scores in particular brain regions.

The device 16 may further include an input arrangement 26 (e.g., touch screen/pad, keypad, mouse, etc.) for configuring the components/settings of the device 16 and/or for manipulating the EEG data and/or the data shown on the output arrangement 24. To communicate with these and any other peripheral components, the device 16 preferably includes suitable hardware ports and software drivers or a wireless communication arrangement (e.g., Bluetooth).

Furthermore, as would be understood by those of skill in the art, the electrical signals may be contaminated by voltages associated with body movements (e.g., eye movements), abnormal physiological events, etc. These contaminating voltages are typically greater than those created by brain activity and thus, algorithms may be used to minimize the impact of such contaminating events. For example, where brain activity is detected through EEG, an updateable voltage threshold may be computed continuously for the EEG channel (or separately for each channel in the case of more than one EEG channel) by calculating a root mean square (rms) voltage for a sliding 20-second window and multiplying the rms-voltage by a constant selected so that the rms-voltage is approximately 0.2 standard deviations of the amplitude of the electrical signals. Segments of the electrical signals containing voltages larger than the selected threshold are considered artifacts and the EEG may be filtered to remove these artifacts. In the exemplary embodiment, the threshold is a multiple of the rms-voltage equal to approximately six (6) times the standard deviation of the amplitude. In other exemplary embodiments, the threshold may be a static value which is a maximum value expected to be generated by brain activity (i.e., a value above which all voltages are considered to result from artifacts). After the EEG has been filtered, remaining segments of the electrical signals are assumed to be substantially artifact-free and these remaining segments are compiled to form a continuous, artifact-free EEG sample.

In other embodiments, across a sliding window of 20 seconds, a continuously updated value is computed of the means (M) and standard deviations (SD) of amplitude (V), slope (V′, or first difference), sharpness (V″, or second difference) at every sample point in every electrode channel. At any time point, data which exceeds M+2.6 SD is considered to reflect an artifact and is rejected from further processing.

In a preferred embodiment, events producing signals that are provisionally considered as artifacts on the basis on any of the previously described criteria may be considered as putative epileptiform events (EE) of clinical significance and are subjected to evaluation by a computerized pattern recognition algorithm serving as an “EE detector”. In this embodiment, the number of EE events detected in each channel is considered to be an additional QEEG feature and is included separately among the items in the pre- and post-treatment profiles to evaluate the efficacy of the treatment.

Measurements of the brain wave activity of the subject 20 should be reliably replicable. Ideally, the pre- and post-treatment profiles are each evaluated for test-retest reliability using a t-test or another statistical method that measures reliability. Preferably, odd and even split halves may be constructed by assigning intervals alternately to two interlocked, but independent samples, each containing, for example, two minutes of artifact-free data consisting of 48 segments each 2.5 seconds in duration. The significance of differences between the split halves is computed as the t-test for each of the extracted features. The t-test provides an accurate indication of replicablity and can be applied at each time point t as follows:

$$t = \frac{( {V}_{1} - {V}_{2} )}{{\vert {F}_{V 1}^{2} + {F}_{V 2}^{2} \vert}^{1 / 2}}$$

where V1=the mean voltage of the odd half
V2=the mean voltage of the even half
FV12=the variance of the odd half
and FV22=the variance of the even half

The t-value calculated using the formula above is compared to a predetermined t-value which is a function of the number of samples in each half and a risk factor selected by the medical personnel. The t-test fails when the calculated t-value exceeds the predetermined t-value. This indicates that the difference between the two halves is statistically significant and thus unreliable. If either the pre- or post-treatment profiles fails the t-test, the EEG data is collected again under similar conditions until the profile passes t-test.

Assuming the profiles have passed the t-test, the EEG data is evaluated using a quantitative assessment of expected normality (e.g., the population norm) of the signals such as “Neurometrics” (the computerized quantitative analysis of brain electrical activity). In Neurometric analysis, features are extracted from quantitative electroencephalogram (QEEG), transformed to obtain Gaussianity, compared to expected normative values (e.g., the self and/or population norms) and expressed as standard deviations from the reference norm. The results may be displayed, for example, as color-coded topographic probability maps of brain function. Utilizing these methods greatly enhances the sensitivity, specificity and clinical utility of such data.

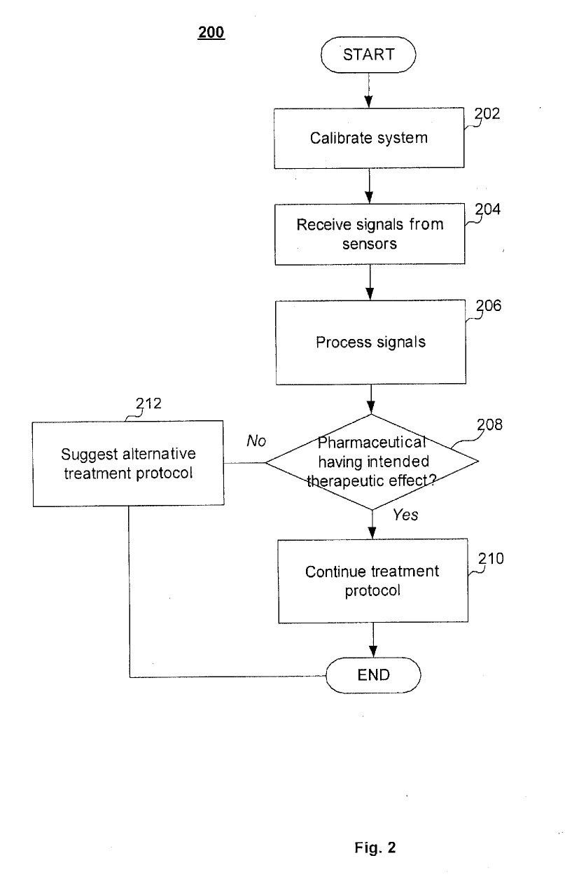

An exemplary embodiment of a method 200 for assessing therapeutic agent efficacy according to the present invention is shown in FIG. 2. In step 202, the system 1 is initialized and calibrated. The device 16 and the output and input arrangements 24, 26 are powered and configured for the brain wave analysis method in accordance with the methodology described herein. The system 1 may be configured based on subject data, e.g., height, weight, age, medical history, etc., which may be used to determine the efficacy of a therapeutic agent (or combination of therapeutic agents) administered to the subject 20 and, optionally, to suggest one or more directions for improving the method of treatment (e.g., other agents, different dosages/time frames of administration, etc.) via a comparison to the treatment data, as will be explained below.

In step 204, the device 16 receives signals corresponding to brain activity of the subject 20 (e.g., electrical signals from electrodes 8 attached to the scalp of the subject 20) and in step 206, the signals are processed by the device 16 in the manner described above. That is, QEEG of the subject 20 are used to generate data corresponding to the brain activity of the subject 20 with this data being filtered and smoothed to reduce the effects of ambient noise and artifacts.

In step 208, the EEG data is compared to reference norms and a determination is made as to whether the agent(s) administered is (are) having a desired therapeutic effect on the disorder, e.g., returning the subject 20 to normal EEG or to the EEG of an individual with a more manageable form of the disorder. In the exemplary embodiment, a reference baseline corresponding to EEG data of an individual with similar age, ethnicity, medical background, etc. (or corresponding to an amalgam baseline corresponding to a group os such similar individuals) may be selected from the population norms in the database 6. As previously discussed, the database 6 and/or the memory of the device 16 may store self norm data for the subject 20, i.e., EEG data of the subject 20 obtained prior to administration of the agent(s) and/or during periods when the subject is substantially symptom free. This EEG data is compared to the population norm and/or the self norm to determine what, if any, effect the agent has had in returning the subject 20 to normal EEG, e.g., the population norm. Additionally, the EEG data may be mapped onto brain activity data for individuals similar to the subject 20 suffering from the same or similar disorders. In this manner, the effect of the agent on the subject 20 may be determined relative to its effect on similar individuals.

In step 210, the comparison of the EEG data to the reference norm indicates that the agent(s) is (are) having the intended therapeutic effect and the treatment protocol is continued for the subject 20. In step 212, the comparison of the EEG data to the reference norm indicates that the disorder is not being alleviated by the agent(s). In this case, based on his experience and/or the symptoms exhibited by or described by the subject 20 after administration of the agent(s), the physician may revise the treatment protocol by switching to a different agent(s), adjusting dosage, etc. Additionally, the EEG data may be mapped onto the treatment data. That is, the EEG data may be matched to EEG data of one or more individuals similar to the subject 20 suffering from a similar disorder to output an alternative treatment protocol.

The present invention provides an objective neurobiological basis for management of developmental, neurological and psychiatric disorders through the application of therapeutic agents with the device 16 providing physicians with objective evidence as to the efficacy of therapeutic agents. Those of skill in the art will understand that the device 16, in other exemplary embodiments, may be used for diagnosis and/or prescriptive intervention. In the former case, for example, the EEG data may be compared to a database of brain activity data profiles for individuals suffering from different developmental, neurological and psychiatric disorders, and combinations thereof. Each of the profiles may be associated with a treatment protocol used to treat the disorder of the corresponding individual. The profiles may further include, for example, a list of agents administered to the individual, dosages, subsequent EEG data collect after predefined time intervals, etc. By matching the EEG data of the subject 20 to one or more of these profiles, the physician may create a treatment protocol for the subject 20 based on the treatment protocols associated with the profiles.

It will be apparent to those skilled in the art that various modifications and variations can be made in the structure and the methodology of the present invention, without departing from the spirit or scope of the invention. Thus, it is intended that the present invention cover the modifications and variations of this invention provided they come within the scope of the appended claims and their equivalents.

What is claimed is:

1. A method for assessing an effect of a therapeutic agent, comprising the steps of:
detecting brain electrical activity of a subject to generate a first set of brain wave data;
extracting from the first set of brain wave data first data features sensitive to a neurological disorder; and
comparing the first data features to control data to define a baseline profile of brain electropathophysiology; and
computing one of a first classifying score and a first discriminant score based on the baseline profile to estimate a probability that the baseline profile corresponds to a predetermined pathophysiological condition.

2. The method according to claim 1, further comprising:
administering a dosage of a therapeutic agent to the subject; and
after a predetermined time interval has elapsed since the administration of the dosage, detecting brain electrical activity of the subject to generate a second set of brain wave data, the predetermined time interval being determined based on pharmocokinetic properties of the therapeutic agent.

3. The method according to claim 2, further comprising:
subjecting the second set of brain wave data to spectral analysis to select second data features sensitive to a neurological disorder; and
comparing the second data features to control data to define a post-treatment profile of brain electropathophysiology; and
computing one of a second classifying score and a second discriminant score based on the post-treatment profile to evaluate an effect of the therapeutic agent.

4. The method according to claim 1, wherein the control data includes population norm data, the method further comprising:
compiling first population brain wave data from each of a first plurality of individuals, the first population brain wave data including data corresponding to the first data features;
sorting the first population brain wave data based on criteria including at least one of age, medical history, gender and ethnicity of the first plurality of individuals; and
selecting portions of the first population brain wave data for use as the population norm data based on a comparison of the criteria and corresponding characteristics of the subject.

5. The method according to claim 4, wherein the first plurality of individuals are selected from a group including one of individuals not suffering from the neurological disorder, individuals with a form of the neurological disorder more manageable than that of the subject and individuals substantially free of symptoms of the neurological disorder.

6. The method according to claim 4, further comprising:
compiling second population brain wave data from each of a second plurality of individuals, the second population brain wave data including data corresponding to the first data features;
sorting the second population brain wave data based on second criteria including at least one of a therapeutic agent used to treat the disorder of the corresponding individual and an observed therapeutic effect of the agent in treating the disorder of the corresponding individual;
selecting portions of the second population brain wave data for use as the population norm data based on a comparison of the criteria and corresponding characteristics of the subject;
comparing of at least one of (i) the neurological disorder of the subject and a neurological disorder of a respective one of the individuals of the second plurality of individuals; and (ii) the therapeutic agent and a treatment regimen of the respective one of the individuals of the second plurality of individuals; and
analyzing a therapeutic effect of the therapeutic agent based on an observed therapeutic effect of the treatment regimen of the respective one of the individuals of the second plurality of individuals.

7. The method according to claim 6, further comprising generating an updated treatment protocol for the subject based on the analysis of the therapeutic effect, the updated treatment protocol including at least one of (i) a suggested further therapeutic agent, (ii) a suggested combination of therapeutic agents; and (iii) an adjusted dosage of the therapeutic agent.

8. The method according to claim 1, wherein the data features are extracted from the first set of brain wave data using spectral analysis.

9. A device for assessing a treatment protocol, comprising:
a brain wave detection apparatus gathering a first set of brain wave data corresponding to electrical activity of a brain of a subject; and
a processor extracting from the first set of brain wave data first data features sensitive to a neurological disorder and comparing the first data features to control data to define a baseline profile of brain electropathophysiology and computing one of a first classifying score and a first discriminant score based on the baseline profile to estimate a probability that the baseline profile corresponds to a predetermined pathophysiological condition.

10. The device according to claim 9, wherein the brain wave detection apparatus includes a plurality of EEG electrodes.

11. The device according to claim 9, further comprising a display screen displaying one of the first set of brain wave data, the first data features and the control data.

12. The device according to claim 9, wherein the processor establishes a self norm for the subject based on the first data features prior to administration of a therapeutic agent.

13. The device according to claim 12, wherein the processor compares the self norm to a second set of brain wave data extracted from data gathered after administration of the therapeutic agent to determine an effect of the therapeutic agent.

14. The device according to claim 9, further comprising an interface for coupling the processor to a first database including population brain wave data from each of a plurality of subjects, the processor selecting a portion of the population brain wave data for use as the population norm based on criteria including at least one of age, medical history, gender and ethnicity.

15. The device according to claim 14, wherein none of the plurality of subjects suffers from the neurological disorder.

16. The device according to claim 15, wherein one of the first database and a second database includes further population brain wave data from each of a further plurality of subjects, the processor selecting a portion of the further population brain wave data for use as treatment data corresponding to a suggested treatment protocol based on further criteria including at least one of a neurological disorder of a corresponding subject of the further plurality of subjects, a therapeutic agent administered to the corresponding subject and an observed therapeutic effect of the agent on the corresponding subject.

17. The device according to claim 16, wherein the processor generates the treatment data by analyzing the portion of the further population brain wave data based on a similarity between at least one of (i) the neurological disorder of the subject and the neurological disorder of the corresponding subject; and (ii) the therapeutic agent administered to the subject and the therapeutic agent administered to the corresponding subject, the processor analyzing a therapeutic effect of the therapeutic agent administered to the subject based on a corresponding therapeutic effect of the therapeutic agent administered to the corresponding subject.

18. The device according to claim 17, wherein the suggested treatment protocol includes at least one of (i) a recommended further therapeutic agent; (ii) a recommended combination of therapeutic agents; and (iii) an adjusted dosage of the therapeutic agent.

19. The device according to claim 10, wherein the processor analyzes the first set of brain wave data using at least one of a Fast Fourier Transform (FFT), an Inverse Fast Fourier Transform (IFFT), wavelet analysis, principal component analysis, a logistic regression, a microstate analysis and wavelet demising.

20. A method for formulating a treatment protocol, comprising:
detecting brain wave activity of a subject to generate a first set of brain wave data;
generating a baseline profile by extracting from the first set of brain wave data first selected data features sensitive to a neurological disorder; and
selecting a therapeutic agent based on a comparison of the subject to a plurality of individuals who responded favorably to the therapeutic agent.

21. The method according to claim 20, wherein the individuals include at least one having data features similar to those of the baseline profile and diagnosed with a neurological disorder diagnosed for the subject.

22. The method according to claim 21, wherein the data feature similarity is determined by generating a discriminant score as a function of the standardized score and the individual profiles.

23. The method according to claim 21, wherein the baseline profile is stored in a machine-readable medium and accessed using a unique identifier.
